(19) United States
(12) Patent Application Publication
(10) Pub. No.: US 2025/0171518 A1
(43) Pub. Date: May 29, 2025

(54) METHODS FOR PREPARING CELLS FOR ADOPTIVE T CELL THERAPY

(71) Applicant: City of Hope, Duarte, CA (US)

(72) Inventors: Stephen J. Forman, Duarte, CA (US); Xiuli Wang, Temple City, CA (US)

(21) Appl. No.: 18/754,973

(22) Filed: Jun. 26, 2024

Related U.S. Application Data
(63) Continuation of application No. 16/989,686, filed on Aug. 10, 2020, now abandoned.
Application No. 16/989,686 is a continuation of application No. 15/773,807, filed on May 4, 2018, now abandoned.
(60) Provisional application No. 62/251,620, filed on Nov. 5, 2015.

(51) Int. Cl. C07K 14/705; A61K 40/11; A61K 40/31; A61K 40/42; C07K 14/725; C07K 14/73; C07K 16/28; C07K 16/30; C12N 5/0783

(52) U.S. Cl. CPC C07K 14/70521; A61K 40/11; A61K 40/31; A61K 40/4211; C07K 14/7051; C07K 14/70514; C07K 14/70517; C07K 14/70578; C07K 16/2803; C07K 16/30; C12N 5/0636; A61K 2239/31; A61K 2239/38; A61K 2239/48; C07K 2317/622; C07K 2319/00; C07K 2319/02; C07K 2319/03; C12N 2501/2302; C12N 2501/2315; C12N 2501/727; C12N 2501/999; C12N 2510/00

(57) ABSTRACT

An improved method for preparing T cell populations expressing a chimeric antigen receptor is described.

### CLAIM OF PRIORITY

This application is a continuation of U.S. patent application Ser. No. 16/989,686, filed Aug. 10, 2020, which is a continuation of U.S. patent application Ser. No. 15/773,807, filed May 4, 2018, which is the National Stage Application under 35 U.S.C. § 371 and claims the benefit of International Application No. PCT/US2016/060478, filed Nov. 4, 2016, which claims the benefit of U.S. Provisional Application Ser. No. 62/251,620, filed on Nov. 5, 2015. The entire contents of the foregoing are incorporated herein by reference.

### SEQUENCE LISTING

This application contains a Sequence Listing that has been submitted electronically as an XML file named “40056-0027003_ST26_SL.XML.” The XML file, created on Jun. 26, 2024, is 23,004 bytes in size. The material in the XML file is hereby incorporated by reference in its entirety.

### BACKGROUND

Tumor-specific T cell based immunotherapies, including therapies employing engineered T cells, have been investigated for anti-tumor treatment. In some cases, the T cells used in such therapies do not remain active in vivo for a long enough period. Therefore, there is a need in the art for tumor-specific cancer therapies with longer term, more potent anti-tumor functioning.

Adoptive T cell therapy (ACT) utilizing chimeric antigen receptor (CAR) engineered T cells may provide a safe and effective way to treat various cancers, since CAR T cells can be engineered to specifically recognize antigenically-distinct tumor populations (Cartellieri et al. 2010 J Biomed Biotechnol 2010:956304; Ahmed et al. 2010 Clin Cancer Res 16:474; Sampson et al. 2014 Clin Cancer Res 20:972; Brown et al. 2013 Clin Cancer Res 2012 18:2199; Chow et al. 2013 Mol Ther 21:629).

### SUMMARY

Described herein are methods for providing improved T cell populations for use in various types of T cell therapy. The methods entail culturing and/or expanding T cells, e.g., CAR-expressing T cells, in the presence of an Akt inhibitor, e.g., Akt Inhibitor VIII (CAS No. 612847 Sep. 3). T cell types that can be cultured and/or expanded in the presence of an Akt inhibitor include: CAR T cells, Tumor Infiltrating lymphocytes (“TIL”), TCR-engineered T cells, or T cell clones. The T cell populations can include: PBMC, isolated central memory T cells, isolated naïve T cells, isolated stem memory T cells and combinations thereof.

Studies described below demonstrate that the presence of an Akt inhibitor during ex vivo expansion of CAR T cells can significantly improve the anti-tumor activity of the CAR T cells following adoptive transfer.

Akt inhibitors include: the Akt inhibitor is selected from the group consisting of: Akt Inhibitor VIII (1,3-dihydro-1-[1-[[4-(6-phenyl-1H-imidazo[4,5-g]quinoxalin-7-yl)phenyl]methyl]-4-piperidinyl]-2H-benzimidazol-2- one), Akt Inhibitor X (2-chloro-N,N-diethyl-10H-phenoxazine-10-butanamine, monohydrochloride), MK-2206 (8-(4-(1-aminocyclobutyl)phenyl)-9-phenyl-[1,2,4]triazolo[3,4-f][1,6]naphthyridin-3(2H)-one), uprosertib (N-((S)-1-amino-3-(3,4-difluorophenyl)propan-2-yl)-5-chloro-4-(4-chloro-1-methyl-1H-pyrazol-5-yl)furan-2-carboxamide), ipatasertib ((S)-2-(4-chlorophenyl)-1-(4-((5R,7R)-7-hydroxy-5-methyl-6,7-dihydro-5H-cyclopenta[d]pyrimidin-4-yl)piperazin-1-yl)-3- (isopropylamino)propan-1-one), AZD 5363 (4-Piperidinecarboxamide, 4-amino-N-[(1S)-1-(4-chlorophenyl)-3-hydroxypropyl]-1-(7H-pyrrolo[2,3-d]pyrimidin-4-yl)), perifosine, GSK690693, GDC-0068, tricirbine, CCT128930, A-674563, PF-04691502, AT7867, miltefosine, PHT-427, honokiol, triciribine phosphate, and KP372-1A (10H-indeno[2,1-e]tetrazolo[1,5-b][1,2,4]triazin-10-one), Akt Inhibitor IX (CAS 98510-80-6).

Additional Akt inhibitors include: ATP-competitive inhibitors, e.g. isoquinoline-5-sulfonamides (e.g., H-8, H-89, NL-71-101), azepane derivatives (e.g., (-)-balanol derivatives), aminofurazans (e.g., GSK690693), heterocyclic rings (e.g., 7-azaindole, 6-phenylpurine derivatives, pyrrolo[2,3-d]pyrimidine derivatives, CCT128930, 3-aminopyrrolidine, anilinotriazole derivatives, spiroindoline derivatives, AZD5363, A-674563, A-443654), phenylpyrazole derivatives (e.g., AT7867, AT13148), thiophenecarboxamide derivatives (e.g., Afuresertib (GSK2110183), 2-pyrimidyl-5-amidothiophene derivative (DC120), uprosertib (GSK2141795); Allosteric inhibitors, e.g., 2,3-diphenylquinoxaline analogues (e.g., 2,3-diphenylquinoxaline derivatives, triazolo[3,4-f][1,6]naphthyridin-3(2H)-one derivative (MK-2206)), alkylphospholipids (e.g., Edelfosine (1-O-octadecyl-2-O-methyl-rac-glycero-3-phosphocholine, ET-18-OCH3) ilmofosine (BM 41.440), miltefosine (hexadecylphosphocholine, HePC), perifosine (D-21266), erucylphosphocholine (ErPC), erufosine (ErPC3, erucylphosphohomocholine), indole-3-carbinol analogues (e.g., indole-3-carbinol, 3-chloroacetylindole, diindolylmethane, diethyl 6-methoxy-5,7-dihydroindolo [2,3-b]carbazole-2,10-dicarboxylate (SR13668), OSU-A9), Sulfonamide derivatives (e.g., PH-316, PHT-427), thiourea derivatives (e.g., PIT-1, PIT-2, DM-PIT-1, N-[(1-methyl-1H-pyrazol-4-yl)carbonyl]-N′-(3-bromophenyl)-thiourea), purine derivatives (e.g., Triciribine (TCN, NSC 154020), triciribine mono-phosphate active analogue (TCN-P),4-amino-pyrido[2,3-d]pyrimidine derivative API-1, 3-phenyl-3H-imidazo[4,5-b]pyridine derivatives, ARQ 092), BAY 1125976, 3-methyl-xanthine, quinoline-4-carboxamide, 2-[4-(cyclohexa-1,3-dien-1-yl)-1H-pyrazol-3-yl]phenol, 3-oxo-tirucallic acid, 3α- and 3β-acetoxy-tirucallic acids, acetoxy-tirucallic acid; and irreversible inhibitors, e.g., natural products, antibiotics, Lactoquinomycin, Frenolicin B, kalafungin, medermycin, Boc-Phe-vinyl ketone, 4-hydroxynonenal (4-HNE), 1,6-naphthyridinone derivatives, and imidazo-1,2-pyridine derivatives, and

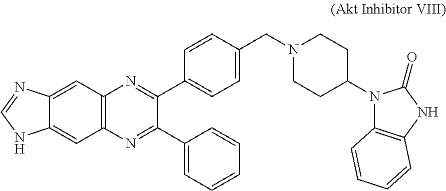 [SMILES: O=c1[nH]c2ccccc2n1C1CCN(Cc2ccc(-c3nc4cc5nc[nH]c5cc4nc3-c3ccccc3)cc2)CC1]

The PI3K-Akt-mTOR pathway plays an important role in regulating CD8+ T-cell metabolism and differentiation. The PI3K-Akt pathway is activated in response to T-cell receptor signaling, costimulatory molecules, and cytokine receptors. This leads to activation of the mammalian target of rapamycin (mTOR) complex-1 and cytoplasmic sequestration of Forkhead box protein O1 (Foxo1). It appears that constitutively active Akt, a kinase, induces terminal differentiation. There are three related forms of human Akt: Akt1 (human RAC-alpha serine/threonine-protein kinase; GenBank® Reference: NP_001014431), Akt2 (human RAC-beta serine/threonine-protein kinase isoform 2; GenBank® Reference: NP_001229956) and Akt3 (RAC-gamma serine/threonine-protein kinase isoform 2; GenBank® Reference: NP_001193658). The three forms are also known as protein kinase B isoforms PKB α, β, γ). Useful Akt inhibitors inhibit at least one of the three forms, preferably with an IC50 that is less than 1000 nM. In some cases, the inhibitor inhibits two or more forms, e.g., Akt 1 and Akt 2 cach with an IC50 that is less than 1000 nM.

The T cell populations that can be treated as described herein harbor an expression vector (e.g., a viral expression vector) encoding a CAR which comprises an extracellular domain, a transmembrane region and an intracellular signaling domain. The extracellular domain is made up of a ligand that binds a target, e.g., CD19 or HER2, and, optionally, a spacer, comprising, for example a portion human Fc domain. The transmembrane portion includes a CD4 transmembrane domain, a CD8 transmembrane domain, a CD28 transmembrane domain, a CD3 transmembrane domain or a 4IBB transmembrane domain. The intracellular signaling domain includes the signaling domain from the zeta chain of the human CD3 complex (CD3ζ) and one or more costimulatory domains, c.g., a 4-1BB costimulatory domain. The extracellular domain enables the CAR, when expressed on the surface of a T cell, to direct T cell activity to those cells expressing the target. The inclusion of a costimulatory domain, such as the 4-1BB (CD137) costimulatory domain in series with CD3ζ in the intracellular region enables the T cell to receive co-stimulatory signals. T cells, for example, patient-specific, autologous T cells can be engineered to express the CARs described herein and the engineered cells can be expanded and used in ACT. Various T cell subsets can be used. In addition, the CAR can be expressed in other immune cells such as NK cells. Where a patient is treated with a T cell population expressing a CAR described herein the cell can be an autologous or allogenic T cell. In some cases, the cells used are CD4+ and CD8+ central memory T cells (T_{CM}), which are CD45RO+CD62L+, and the use of such cells can improve long-term persistence of the cells after adoptive transfer compared to the use of other types of patient-specific T cells.

The costimulatory domain can be selected from, for example, the group consisting of: a CD28 costimulatory domain or a variant thereof having 1-10 (e.g., 1 or 2) amino acid modifications, a 4-IBB costimulatory domain or a variant thereof having 1-10 (e.g., 1 or 2) amino acid modifications and an OX40 costimulatory domain or a variant thereof having 1-10 (e.g., 1 or 2) amino acid modifications. In certain embodiments, a 4IBB costimulatory domain or a variant thereof having 1-10 (e.g., 1 or 2) amino acid modifications in present.

The CAR can comprise: two different costimulatory domains selected from the group consisting of: a CD28 costimulatory domain or a variant thereof having 1-10 (e.g., 1 or 2) amino acid modifications, a 4IBB costimulatory domain or a variant thereof having 1-10 (e.g., 1 or 2) amino acid modifications and an OX40 costimulatory domain or a variant thereof having 1-10 (e.g., 1 or 2) amino acid modifications; two different costimulatory domains selected from the group consisting of: a CD28 costimulatory domain or a variant thereof having 1-2 amino acid modifications, a 4IBB costimulatory domain or a variant thereof having 1-2 amino acid modifications and an OX40 costimulatory domain or a variant thereof having 1-2 amino acid modifications; human IL-13 or a variant thereof having 1-2 amino acid modifications; a transmembrane domain selected from: a CD4 transmembrane domain or variant thereof having 1-2 amino acid modifications, a CD8 transmembrane domain or variant thereof having 1-2 amino acid modifications, a CD28 transmembrane domain or a variant thereof having 1-2 amino acid modifications, and a CD3ζ transmembrane domain or a variant thereof having 1-2 amino acid modifications; a costimulatory domain; and CD3ζ signaling domain of a variant thereof having 1-2 amino acid modifications; a spacer region located between the IL-13 or variant thereof and the transmembrane domain (e.g., the spacer region comprises an amino acid sequence selected from the group consisting of SEQ ID NO:4, 14-20, 50 and 52); the spacer comprises an IgG hinge region; the spacer region comprises 10-150 amino acids; the 4-1BB signaling domain comprises the amino acid sequence of SEQ ID NO:6; the CD3ζ signaling domain comprises the amino acid sequence of SEQ ID NO:7; and a linker of 3 to 15 amino acids that is located between the costimulatory domain and the CD3ζ signaling domain or variant thereof. In certain embodiments where there are two costimulatory domains, one is a 4-IBB costimulatory domain and the other a costimulatory domain selected from: CD28 and CD28gg.

### DESCRIPTION OF DRAWINGS

FIG. 1: An Akt inhibitor did not compromise the CD19CAR T cell expansion in vitro. Total cell number is plotted as a function of the number of days of expansion. CD8+ T cells were selected, activated with CD3/CD28 beads, and transduced with CD19CAR lentivirus. The transduced T cells were maintained in the presence of IL-2 50U/mL and Akt inhibitor (1 uM/mL) (Akt inhibitor VIII, CAS 612847-09-3, a cell-permeable, reversible & selective inhibitor of Akt1/Akt2 (IC50=58 nM and 210 nM for Akt1 & Akt2, respectively); EMD Millipore). The cultures without Akt inhibitor were used as controls. Total viable cells were measured every other day.

FIG. 2: An Akt inhibitor did not inhibit the effector function of CD19CAR T cells. CD8+CD19CAR expression T cells were expanded in the presence or absence of Akt inhibitor VIII for 21 days. A 107a degranulation assay was performed after overnight co-culturing of the CD19CAR T cells with CD19+LCL cells. OKT3 expressing LCL were used as positive control and CD19 negative AML cells KG1a were used as negative control.

FIG. 3: Higher CD62L expression on the Akt inhibitor treated CD19CAR T cells. CD8+ T cells were selected, activated with CD3/CD28 beads, and transduced with CD19CAR lentivirus. The transduced T cells were maintained in the presence of IL-2 50U/mL and Akt inhibitor VIII (1 uM/mL) (Akt inhibitor VIII, from EMD Millipore). The cultures without Akt inhibitor were used as controls. CAR expression was detected with Erbitux for EGFRt. % CAR+CD62L+double positive cells are depicted.

FIGS. 4A-4B: Akt inhibitor treated CD19CAR T cells exhibited central memory characteristics CD8+ T cells were selected, activated with CD3/CD28 beads, and transduced with CD19CAR lentivirus. The transduced T cells were maintained in the presence of IL2 50U/mL and Akt inhibitor VIII (1 uM/mL) Akt. The cultures without Akt inhibitor were used as controls. CD28 and CD62L expression are presented on gated CAR positive population.

FIGS. 5A-5B: Ex vivo Akt inhibition (Akti) generates potent CD19CAR T cells for adoptive therapy. CD19+ acute lymphoid leukemia cells (0.5×10⁶; SupB15) engineered to express firefly luciferase were inoculated intravenously into NSG mice. At 5 days post tumor engraftment, 2×10⁶ CD19 re-directed CD8+ T cells (CD19CAR) that were expanded in vitro in the presence of Akt inhibitor VIII were intravenously injected into tumor bearing mice. Mice that received no T cells, non-transduced T cells (Mock), and CD19CAR T cells that were not treated with Akt inhibitor during in vitro expansion were used as controls. Tumor signals post CD19CAR T cell infusion were monitored by biophotonic imaging.

FIGS. 6A-6B: Akt inhibition promotes the generation of memory CD19 CAR T cells from different T cell subsets. (A) Bulk T cells (PBMC), purified central memory T cells (T_{CM}), and purified naïve/memory T cells (naïve T cells, central memory T cells and stem memory T cells (T_{N}, T_{CM}, and T_{SCM})) were transduced with lentivirus encoding second generation CD19 CAR vector and expanded in a medium containing 50U/L rhIL2, in the presence and absence of 1 μM Akt inhibitor VIII for 17-21 days. Resultant CD19 CAR T cells were stained with biotinylated Erbitux (cetuximab), followed by streptavidin-PE for CAR detection and antibodies against CD62L. Percentages of CAR+CD62L+ cells are depicted on the basis of the gating of isotype-stained cells. (B) Percentages of CD62L+CD28+ T cells after gating on CAR+CD8+ from six lines of CD19 CAR T cells derived from two different donors are presented. For both donors, PBMC, T_{CM}, and T_{N}/T_{CM}/T_{SCM} cell populations were prepared, transduced with the lentivirus encoding the CD19 CAR and then expanded in the absence or presence of Akt inhibitor VIII.

### DETAILED DESCRIPTION

Described are methods for preparing populations of T cells expressing a CAR or some other T cells receptor and having improved anti-tumor activity. The method entails contacting the cells with an inhibitor of Akt, e.g., during culturing and expansion of the T cell receptor expressing T cell population.

A chimeric antigen (CAR) is a recombinant biomolecule that contains, at a minimum, an extracellular recognition domain, a transmembrane region, and an intracellular signaling domain. The term “antigen,” therefore, is not limited to molecules that bind antibodies, but to any molecule that can bind specifically to a target. For example, a CAR can include a ligand that specifically binds a cell surface receptor. The extracellular recognition domain (also referred to as the extracellular domain or simply by the recognition element which it contains) comprises a recognition element that specifically binds to a molecule present on the cell surface of a target cell. The transmembrane region anchors the CAR in the membrane. The intracellular signaling domain comprises the signaling domain from the zeta chain of the human CD3 complex and optionally comprises one or more costimulatory signaling domains. CARs can both to bind antigen and transduce T cell activation, independent of MHC restriction. Thus, CARs are “universal” immunoreceptors which can treat a population of patients with antigen-positive tumors irrespective of their HLA genotype. Adoptive immunotherapy using T lymphocytes that express a tumor-specific CAR can be a powerful therapeutic strategy for the treatment of cancer.

CAR coding sequences can be produced by any means known in the art, though preferably it is produced using recombinant DNA techniques. Nucleic acids encoding the several regions of the chimeric receptor can be prepared and assembled into a complete coding sequence by standard techniques of molecular cloning known in the art (genomic library screening, PCR, primer-assisted ligation, site-directed mutagenesis, etc.) as is convenient. The resulting coding region is preferably inserted into an expression vector and used to transform a suitable expression host cell line, preferably a T lymphocyte cell line, and most preferably an autologous T lymphocyte cell line.

Various T cell subsets isolated from the patient, including unselected PBMC or enriched CD3 T cells or enriched CD3 or memory T cell subsets, can be transduced with a vector for CAR expression or expression of some other T cells receptor and cultured by the methods described herein. Central memory T cells are one useful T cell subsets. Central memory T cell can be isolated from peripheral blood mononuclear cells (PBMC) by selecting for CD45RO+/CD62L+ cells, using, for example, the CliniMACS® device to immunomagnetically select cells expressing the desired receptors. The cells enriched for central memory T cells can be activated with anti-CD3/CD28, transduced with, for example, a SIN lentiviral vector that directs the expression of a CAR (e.g., a CD19 or HER2 specific CAR) as well as a truncated human CD19 (CD19t), a non-immunogenic surface marker for both in vivo detection and potential ex vivo selection. The activated/genetically modified central memory T cells can be expanded in vitro with IL-2/IL-15 and then cryopreserved.

### Example 1: Construction and Structure of a CD19 CAR

The structure of a useful CD19-specific CAR is described below. The construct, CD19R(EQ)CD28T2AEGFRtepHIV7 is described in detail in WO2011/056894. The CAR sequence includes a sequence targeted to CD19, an IgG4 Fc spacer containing two mutations (L235E; N297Q) that greatly reduce Fc receptor-mediated recognition models, a CD28 transmembrane domain, a costimulatory CD28 cytoplasmic signaling domain, and a CD3ζ cytoplasmic signaling domain. A T2A ribosome skip sequence separates this CD19(EQ)28ζ CAR sequence from EGFRt, an inert, non-immunogenic cell surface detection/selection marker. This T2A linkage results in the coordinate expression of both CD19(EQ)28ζ and EGFRt from a single transcript.

The CD19(EQ)28Z sequence was generated by fusion of the human GM-CSF receptor alpha leader peptide with CD19 specific scFv, an L235E/N297Q-modified IgG4 Fc hinge (where the double mutation interferes with FcR recognition), CD28 transmembrane, CD28 cytoplasmic signaling domain, and CD3ζ cytoplasmic signaling domain sequences. This sequence was synthesized de novo after codon optimization. The T2A sequence was obtained from digestion of a T2A-containing plasmid. The EGFRt sequence was obtained from that spanning the leader peptide sequence to the transmembrane components (i.e., basepairs 1-972) of a CD19-containing plasmid. All three fragments, 1) CD19(EQ)28Z, 2) T2A, and 3) EGFRt, were cloned into the multiple cloning site of the epHIV7 lentiviral vector.

### Example 2: Construction and Structure of epHIV7 Used for Expression of a CD19-Specific CAR

The vector epHIV7 used for expression of the CAR was produced from pHIV7 vector. Importantly, this vector uses the human EFI promoter to drive expression of the CAR. Both the 5′ and 3′ sequences of the vector were derived from pv653RSN as previously derived from the HXBc2 provirus. The polypurine tract DNA flap sequences (cPPT) were derived from HIV-1 strain pNL4-3 from the NIH AIDS Reagent Repository. The woodchuck post-transcriptional regulatory element (WPRE) sequence was previously described

Briefly, pv653RSN, containing 653 bp from gag-pol plus 5′ and 3′ long-terminal repeats (LTRs) with an intervening SL3-neomycin phosphotransferase gene (Neo), was subcloned into pBluescript, as follows: In Step 1, the sequences from 5′ LTR to rev-responsive element (RRE) made p5′HIV-1 51, and then the 5′ LTR was modified by removing sequences upstream of the TATA box, and ligated first to a CMV enhancer and then to the SV40 origin of replication (p5′HIV-2). In Step 2, after cloning the 3′ LTR into pBluescript to make p3′HIV-1, a 400-bp deletion in the 3′ LTR enhancer/promoter was made to remove cis-regulatory elements in HIV U3 and form p3′HIV-2. In Step 3, fragments isolated from the p5′HIV-3 and p3′HIV-2 were ligated to make pHIV-3. In Step 4, the p3′HIV-2 was further modified by removing extra upstream HIV sequences to generate p3′HIV-3 and a 600-bp BamHI-Sall fragment containing WPRE was added to p3′HIV-3 to make the p3′HIV-4. In Step 5, the pHIV-3 RRE was reduced in size by PCR and ligated to a 5′ fragment from pHIV-3 (not shown) and to the p3′HIV-4, to make pHIV-6. In Step 6, a 190-bp BglII-BamHI fragment containing the cPPT DNA flap sequence from HIV-1 pNL4-3 was amplified from pNL4-3 and placed between the RRE and the WPRE sequences in pHIV6 to make pHIV-7. This parent plasmid pHIV7-GFP (GFP, green fluorescent protein) was used to package the parent vector using a four-plasmid system.

A packaging signal, psi Ψ, is required for efficient packaging of viral genome into the vector. The RRE and WPRE enhance the RNA transcript transport and expression of the transgene. The flap sequence, in combination with WPRE, has been demonstrated to enhance the transduction efficiency of lentiviral vector in mammalian cells.

The helper functions, required for production of the viral vector), are divided into three separate plasmids to reduce the probability of generation of replication competent lentivirus via recombination: 1) pCgp encodes the gag/pol protein required for viral vector assembly; 2) pCMV-Rev2 encodes the Rev protein, which acts on the RRE sequence to assist in the transportation of the viral genome for efficient packaging; and 3) pCMV-G encodes the glycoprotein of the vesiculo-stomatitis virus (VSV), which is required for infectivity of the viral vector.

There is minimal DNA sequence homology between the pHIV7 encoded vector genome and the helper plasmids. The regions of homology include a packaging signal region of approximately 600 nucleotides, located in the gag/pol sequence of the pCgp helper plasmid; a CMV promoter sequence in all three helper plasmids; and a RRE sequence in the helper plasmid pCgp. It is highly improbable that replication competent recombinant virus could be generated due to the homology in these regions, as it would require multiple recombination events. Additionally, any resulting recombinants would be missing the functional LTR and tat sequences required for lentiviral replication.

The CMV promoter was replaced by the EF1α-HTLV promoter (EF1p), and the new plasmid was named epHIV7. The EF1p has 563 bp and was introduced into epHIV7 using NruI and NheI, after the CMV promoter was excised.

The lentiviral genome, excluding gag/pol and rev that are necessary for the pathogenicity of the wild-type virus and are required for productive infection of target cells, has been removed from this system. In addition, the CD19R(EQ)CD28T2AEGFRtepHIV7 vector construct does not contain an intact 3′LTR promoter, so the resulting expressed and reverse transcribed DNA proviral genome in targeted cells will have inactive LTRs. As a result of this design, no HIV-I derived sequences will be transcribed from the provirus and only the therapeutic sequences will be expressed from their respective promoters. The removal of the LTR promoter activity in the SIN vector is expected to significantly reduce the possibility of unintentional activation of host genes.

### Example 3: Production of Vectors for Transduction of Patient T Cells

Vectors for transduction of T cell populations can be prepared as follows. For each plasmid (CD(EQ)BBZ-T2A-CD19t_epHIV7; pCgp; pCMV-G; and pCMV-Rev2), a seed bank is generated, which is used to inoculate the fermenter to produce sufficient quantities of plasmid DNA. The plasmid DNA is tested for identity, sterility and endotoxin prior to its use in producing lentiviral vector.

Briefly, cells are expanded from the 293T working cell (WCB), which has been tested to confirm sterility and the absence of viral contamination. A vial of 293T cells from the 293T WCB is thawed. Cells were grown and expanded until sufficient numbers of cells existed to plate an appropriate number of 10 layer cell factories (CFs) for vector production and cell train maintenance. A single train of cells can be used for production.

The lentiviral vector is produced in sub-batches of up to 10 CFs. Two sub-batches can be produced in the same week leading to the production of approximately 20 L of lentiviral supernatant/week. The material produced from all sub-batches are pooled during the downstream processing phase, in order to produce one lot of product. 293T cells are plated in CFs in 293T medium (DMEM with 10% FBS). Factories are placed in a 37° C. incubator and horizontally leveled in order to get an even distribution of the cells on all the layers of the CF. Two days later, cells are transfected with the four lentiviral plasmids described above using the CaPO₄ method, which involves a mixture of Tris:EDTA, 2M CaCl2, 2×HBS, and the four DNA plasmids. Day 3 after transfection, the supernatant containing secreted lentiviral vectors is collected, purified and concentrated. After the supernatant is removed from the CFs, End-of-Production Cells are collected from each CF. Cells are trypsinized from each factory and collected by centrifugation. Cells are resuspended in freezing medium and cryopreserved. These cells are later used for replication-competent lentivirus (RCL) testing.

To purify and formulate vectors crude supernatant is clarified by membrane filtration to remove the cell debris. The host cell DNA and residual plasmid DNA are degraded by endonuclease digestion (Benzonase®). The viral supernatant is clarified of cellular debris using a 0.45 μm filter. The clarified supernatant is collected into a pre-weighed container into which the Benzonase® is added (final concentration 50 U/mL). The endonuclease digestion for residual plasmid DNA and host genomic DNA is performed at 37° C. for 6 h. The initial tangential flow ultrafiltration (TFF) concentration of the endonuclease-treated supernatant is used to remove residual low molecular weight components from the crude supernatant, while concentrating the virus ˜20 fold. The clarified endonuclease-treated viral supernatant is circulated through a hollow fiber cartridge with a NMWCO of 500 kD at a flow rate designed to maintain the shear rate at ˜4,000 sec-1 or less, while maximizing the flux rate. Diafiltration of the nuclease-treated supernatant is initiated during the concentration process to sustain the cartridge performance. An 80% permeate replacement rate is established, using 4% lactose in PBS as the diafiltration buffer. The viral supernatant is brought to the target volume, representing a 20-fold concentration of the crude supernatant, and the diafiltration is continued for 4 additional exchange volumes, with the permeate replacement rate at 100%.

Further concentration of the viral product was accomplished by using a high speed centrifugation technique. Each sub-batch of the lentivirus is pelleted using a Sorvall RC-26 plus centrifuge at 6000 RPM (6,088 RCF) at 6° C. for 16-20 h. The viral pellet from each sub-batch is then reconstituted in a 50 mL volume with 4% lactose in PBS. The reconstituted pellet in this buffer represents the final formulation for the virus preparation. The entire vector concentration process resulted in a 200-fold volume reduction, approximately. Following the completion of all of the sub-batches, the material is then placed at −80° C., while samples from each sub-batch are tested for sterility. Following confirmation of sample sterility, the sub-batches are rapidly thawed at 37° C. with frequent agitation. The material is then pooled and manually aliquoted in the Class II Type A/B3 biosafety cabinet in the viral vector suite. A fill configuration of 1 mL of the concentrated lentivirus in sterile USP class 6, externally threaded O-ring cryovials is used.

To ensure the purity of the lentiviral vector preparation, it is tested for residual host DNA contaminants, and the transfer of residual host and plasmid DNA. Among other tests, vector identity is evaluated by RT-PCR to ensure that the correct vector is present.

### Example 4: Akt Inhibitor Expanded T Cells Suitable for Use in ACT

T lymphocytes were obtained from healthy subjects by leukopheresis, and CD8+ T cells were isolated magnetically on AutoMACS (Miltenyi). On the day of isolation, 4×10⁶ CD8+ cells in 24 well plate were activated with CD3/CD28 beads at 3:1 (bead:cell) ratio, and transduced with a lentiviral vector encoding the CD19CAR described above at MOI 1.5 in the RPMI1640 medium supplemented with 2 mM L-glutamine, 25 mM HEPES, and 10% heat-inactivated FCS (T cell medium), in presence of IL-2 (50 U/ml) and Akt inhibitor (Akt inhibitor VIII) (1 uM/mL). After 30 minute spinoculation at 567×g at 32° C.±3° C. cultures were then maintained with addition of medium as required to keep cell density between 0.5×10⁶ and 1×10⁶ viable cells/mL with cytokine supplementation of final concentration of 50 U/mL rhIL-2 and Akt inhibitor VIII (1 μM/mL every Monday, Wednesday and Friday of culture. As detailed above, the lentiviral vector also expressed a truncated human epidermal growth factor receptor (huEGFRt) for selection and ablation purposes.

Transduced CD19CAR T cells without Akt inhibitor treatment were used as controls. On day 8 post activation/transduction, beads were removed from the culture using magnet and the engineered CD19CAR T cells were expanded in vitro in RPMI (Irvine Scientific) supplemented with 2 mM L-glutamine, 25 mM HEPES and 10% heat-inactivated FCS (Hyclone) for 21 days before in vitro and in vivo assays.

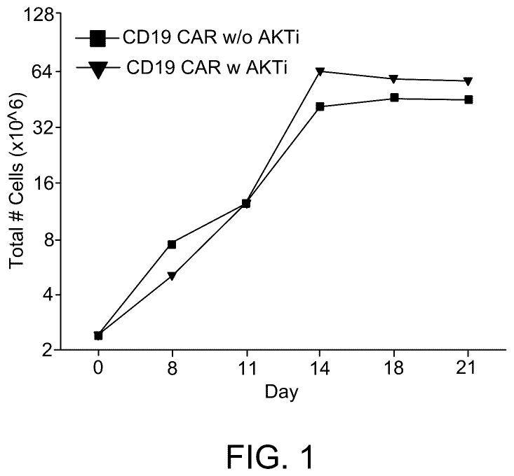

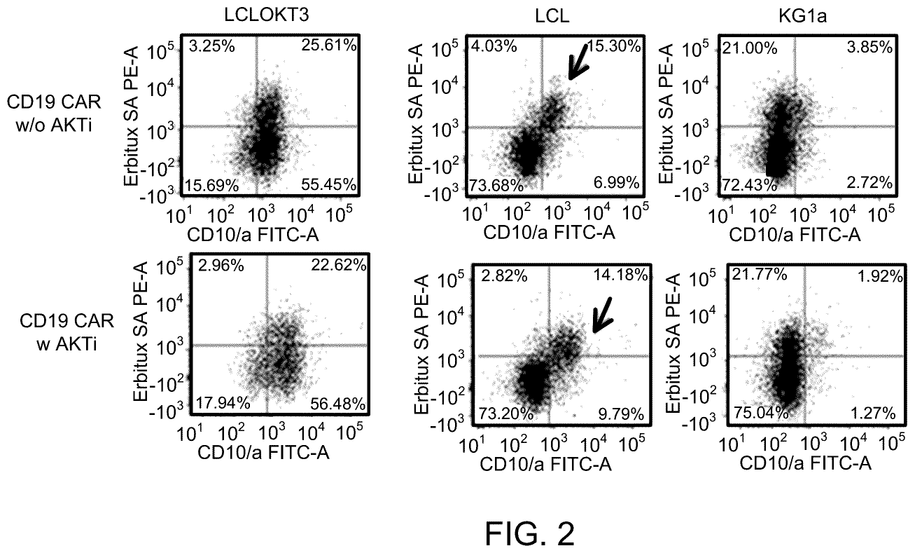

Assessment of proliferation revealed that the presence of Akt inhibitor did not compromise the CD19CAR T cell proliferation and survival in vitro. As shown in FIG. 1, comparable CD19CAR T cell expansion was observed after culturing in the presence or absence of Akt inhibitor. To examiner the potential impact of Akt inhibitor of effector function, engineered CD8+CD19CAR T cells were expanded in the presence or absence of Akt inhibitor for 21 days. A 107a degranulation assay was performed after overnight co-culturing of the CD19CAR T cells with CD19+ LCL cells. OKT3 expressing LCL were used as positive control and CD19 negative AML cells KG1a were used as negative control. The results of this study are presented in FIG. 2 where it can be seen that Akt inhibitor treated cells and untreated cells exhibit equivalent levels of interferon gamma production and CD107a expression upon CD19 antigen stimulation Thus, Akt inhibitor did not appear to dampen the effector function of CD19CAR T cells.

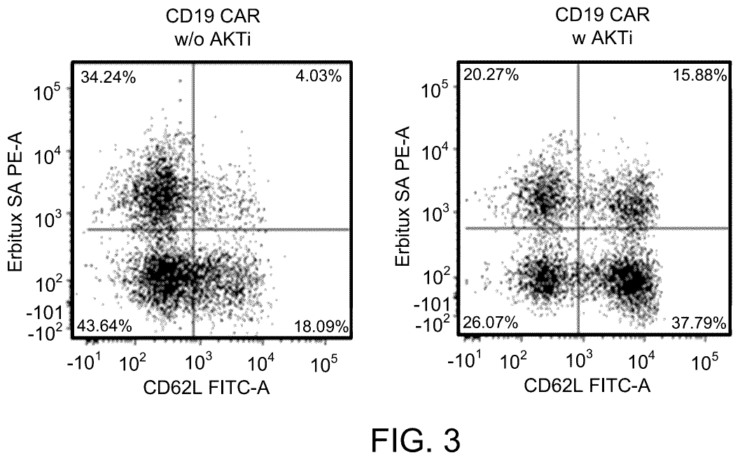

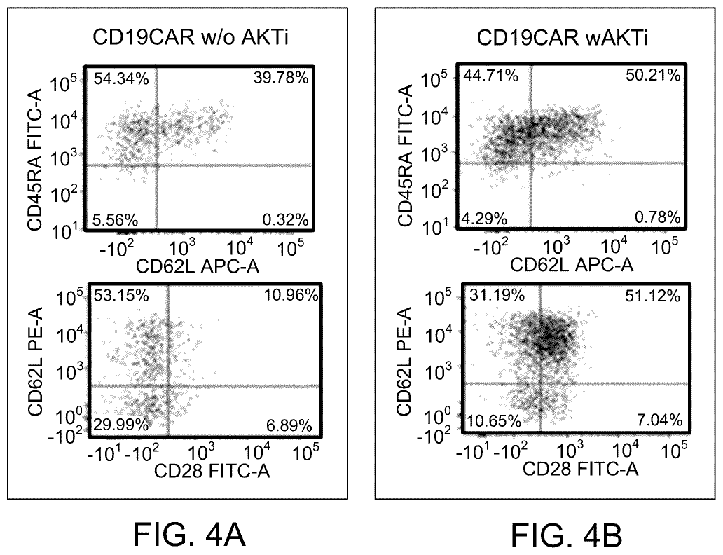

Memory-like phenotype such as CD62L and CD28 expression on CAR T cells is often associated with better antitumor activity in vivo. We therefore characterized the CD19CAR T cells after ex vivo expansion. Briefly, CD8+ T cells were selected, activated with CD3/CD28 beads, and transduced with CD19CAR lentivirus. The transduced T cells were maintained in the presence of IL2 50U/mL and Akt inhibitor VIII. The cultures without Akt inhibitor were used as controls. CAR expression was detected with Erbitux for EGFRt. The results of this study are presented in FIG. 3 (% CAR+CD62L+double positive cells are depicted). We found that 40% of Akt-inhibited CD19CAR T cells expressed CD62L and co-expressed CD28 (FIG. 3 and FIGS. 4A-4B), Meanwhile no exhaustion markers such as KRLG were expressed on the Akt inhibitor treated cells. In contrast, only 10% of control untreated CD19CAR T cells expressed CD62L and they were CD28 negative, indicating that Akt-inhibited CD19CAR T cells may have superior anti-tumor activity following adoptive transfer.

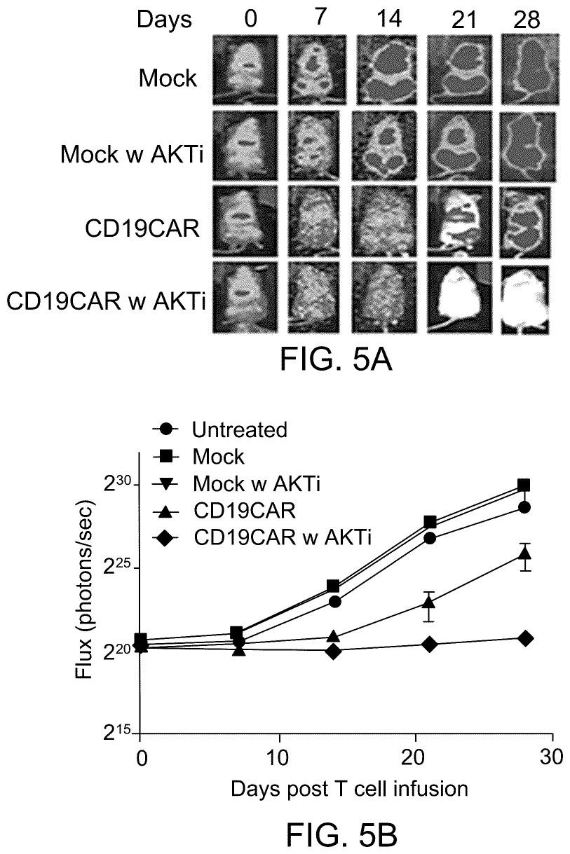

To test the potency of the Akt inhibitor treated CAR T cells, 0.5×10⁶ CD19+ acute lymphoid leukemic cells (SupB15) that were engineered to express firefly luciferase were inoculated intravenously into NOD/Scid IL-2RgammaCnull (NSG) mice. Five days post tumor engraftment, 2×10⁶ CD8+ CD19CAR T cells were intravenously injected into tumor bearing mice. Control mice received either no T cells, non-transduced T cells (Mock), or CD19CAR T cells that were not treated with Akt inhibitor during in vitro expansion. Tumor signals post T cell infusion were monitored by biophotonic imaging. In contrast to the untreated CD19CAR T cells, which exhibited lower and transient anti-tumor activity, Akt-inhibited CD19CAR T cells completely eradicated the CD19+ tumor in all mice (FIGS. 5A-5B), suggesting that inhibition of Akt signaling during the ex vivo priming and expansion gives rise to a CD19CAR T cell population that possesses superior antitumor activity.

### Example 5: Akt Inhibitor Treatment of Central Memory T Cells

Treatment of a CAR T cell population with an Akt inhibitor during expansion and/or activation can be applied to CD8+ cell populations as well as other cell populations, for example, a Central Memory T cell (T_{CM}) population that has been genetically altered to express a CAR.

TcM suitable for expression of a CAR can be prepared as follows. Apheresis products obtained from consented research participants are ficolled, washed and incubated overnight. Cells are then depleted of monocyte, regulatory T cell and naïve T cell populations using GMP grade anti-CD14, anti-CD25 and anti-CD45RA reagents (Miltenyi Biotec) and the CliniMACS™ separation device. Following depletion, negative fraction cells are enriched for CD62L+ T_{CM} cells using DREG56-biotin (COH clinical grade) and anti-biotin microbeads (Miltenyi Biotec) on the CliniMACS™ separation device.

Following enrichment, T_{CM} cells are formulated in complete X-Vivo15 plus 50 IU/mL IL-2 and transferred to a Teflon cell culture bag, where they are stimulated with Dynal ClinEx™ Vivo CD3/CD28 beads. On the day of stimulation, cells are transduced with a vector expressing a desired CAR, for example an HIV7 lentiviral vector at a multiplicity of infection (MOI) of 1.0 to 0.3. Cultures are maintained for up to 21 days with addition of complete X-Vivo15 and IL-2 cytokine as required for cell expansion (keeping cell density between 3×10⁵ and 2×10⁶ viable cells/mL, and cytokine supplementation every Monday, Wednesday and Friday of culture) with periodic addition of an Akt inhibitor. Cells typically expand to approximately 10⁹ cells under these conditions within 21 days. At the end of the culture period cells are harvested, washed twice and formulated in clinical grade cryopreservation medium (Cryostore CS5, BioLife Solutions).

On the day(s) of T cell infusion, the cryopreserved and released product is thawed, washed and formulated for re-infusion. The cryopreserved vials containing the released cell product are removed from liquid nitrogen storage, thawed, cooled and washed with a PBS/2% human serum albumin (HSA) Wash Buffer. After centrifugation, the supernatant is removed and the cells resuspended in a Preservative-Free Normal Saline (PFNS)/2% HSA infusion diluent. Samples are removed for quality control testing.

### Example 6: Akt Inhibitor Treatment Promotes the Generation of Memory T Cells from Different T Cell Subsets

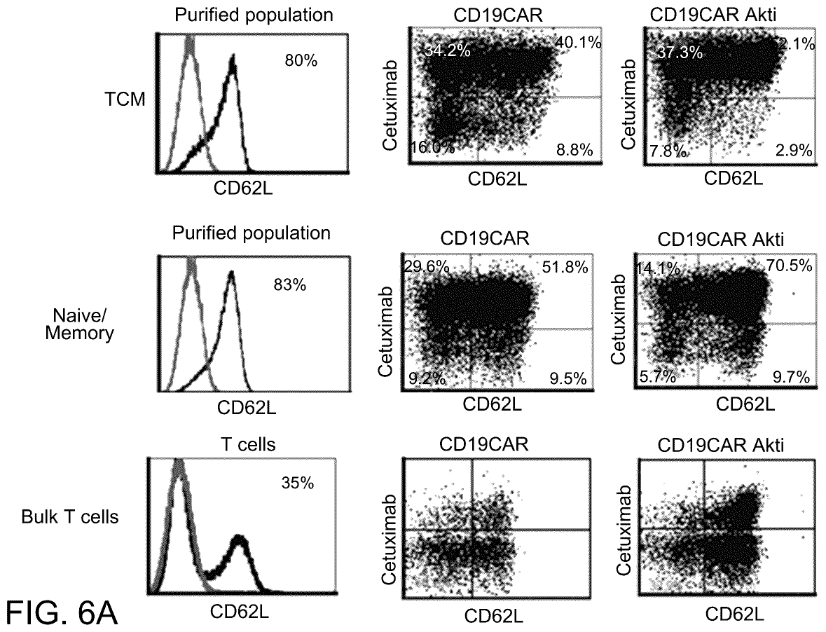

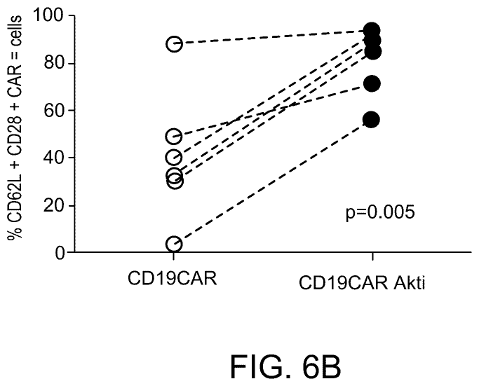

Bulk T cells, purified T_{CM}, purified as described above, and purified naïve/memory T cells (Journal of Immunotherapy 2012 35:689) were transduced with lentivirus encoding the second generation CD19 CAR described above and expanded in a medium containing 50U/L rhIL2, in the presence and absence of 1 μM Akt inhibitor VIII for 17-21 days. Resultant CD19CAR T cells were stained with biotinylated Erbitux (cetuximab), followed by streptavidin-PE for CAR detection and antibodies against CD62L. Cells expressing CD62 represent T_{CM} cells or T_{SCM} cells. Effector T cells do not express CD62L. FIG. 6A presents the results of this analysis where it can be seen the culturing in the presence of an Akt inhibitor increases the percentage of CD62L+ expressing CAR T cells irrespective of whether the starting T cell population was bulk T cells, T_{CM} cells or naïve/memory T cells.

Samples from two donors were used to prepare PBMC, T_{CM}, and T_{N}/T_{CM}/T_{SCM} cell populations. Each of these six cell populations were transduced with the lentivirus encoding the CD19 CAR and then expanded in the absence or presence of Akt inhibitor VIII, as described above, for 17-21 days. As can be seen in FIG. 6B, Akt inhibitor increased the number of CD62L+/CD28+/CAR+ T cells.

### Example 7: Structure of CAR

The methods for producing T cell populations described herein can be used to prepare cells expressing a CAR can be used with any desired CAR. The CAR can include an extracellular domain, a transmembrane region and an intracellular signaling domain. The extracellular domain is made up of a targeting domain which can be a scFv that binds a target, e.g., an scFv that binds HER2 or to some other receptor expressed on tumor cells, or ligand that binds a target, e.g., CD19, and, optionally, a spacer, comprising, for example a portion human Fc domain.

The CAR described herein can include a spacer region located between the targeting domain (i.e., the scFV or ligand) and the transmembrane domain. A variety of different spacers can be used. Some of them include at least portion of a human Fc region, for example a hinge portion of a human Fc region or a CH3 domain or variants thereof. Table 1 below provides various spacers that can be used in the CARs described herein.

TABLE 1
Examples of Spacers

| Name | Length | Sequence |
|---|---|---|
| a3 | 3 aa | AAA |
| linker | 10 aa | GGGSSGGGSG (SEQ ID NO: 2) |
| IgG4 hinge | 12 aa | ESKYGPPCPPCP (SEQ ID NO: 3) |
| (S → P) | | |
| (S228P) | | |
| IgG4 hinge | 12 aa | ESKYGPPCPSCP(SEQ ID NO: 4) |
| IgG4 hinge | 22 aa | ESKYGPPCPPCPGGGSSGGGSG |
| (S228P) + | | (SEQ ID NO: 5) |
| linker | | |
| CD28 hinge | 39 aa | IEVMYPPPYLDNEKSNGTIIHVKGKHL |
| | | CPSPLFPGPSKP (SEQ ID NO: 6) |
| CD8 hinge-48 aa | 48 aa | AKPTTTPAPRPPTPAPTIASQPLSLRPE |
| | | ACRPAAGGAVHTRGLDFACD |
| | | (SEQ ID NO: 7) |
| CD8 hinge-45 aa | 45 aa | TTTPAPRPPTPAPTIASQPLSLRPEACR |
| | | PAAGGAVHTRGLDFACD |
| | | (SEQ ID NO: 8) |
| IgG4(HL-CH3) | 129 aa | ESKYGPPCPPCPGGGSSGGGSGGQPR |
| (includes | | EPQVYTLPPSQEEMTKNQVSLTCLVK |
| S228P | | GFYPSDIAVEWESNGQPENNYKTTPP |
| in hinge) | | VLDSDGSFFLYSRLTVDKSRWQEGNV |
| | | FSCSVMHEALHNHYTQKSLSLSLGK |
| | | (SEQ ID NO: 9) |
| IgG4 | 229 aa | ESKYGPPCPSCPAPEFEGGPSVFLFPPK |
| (L235E, N297Q) | | PKDTLMISRTPEVTCVVVDVSQEDPE |
| | | VQFNWYVDGVEVHQAktKPREEQFQS |
| | | TYRVVSVLTVLHQDWLNGKEYKCKV |
| | | SNKGLPSSIEKTISKAKGQPREPQVYT |
| | | LPPSQEEMTKNQVSLTCLVKGFYPSDI |
| | | AVEWESNGQPENNYKTTPPVLDSDGS |
| | | FFLYSRLTVDKSRWQEGNVFSCSVM |
| | | HEALHNHYTQKSLSLSLGK |
| | | (SEQ ID NO: 10) |
| IgG4(S228P, | 229 aa | ESKYGPPCPPCPAPEFEGGPSVFLFPPK |
| L235E, N297Q) | | PKDTLMISRTPEVTCVVVDVSQEDPE |
| | | VQFNWYVDGVEVHQAKTKPREEQFQ |
| | | STYRVVSVLTVLHQDWLNGKEYKCK |
| | | VSNKGLPSSIEKTISKAKGQPREPQVY |
| | | TLPPSQEEMTKNQVSLTCLVKGFYPS |
| | | DIAVEWESNGQPENNYKTTPPVLDSD |
| | | GSFFLYSRLTVDKSRWQEGNVFSCSV |
| | | MHEALHNHYTQKSLSLSLGK |
| | | (SEQ ID NO: 11) |
| IgG4(CH3) | 107 aa | GQPREPQVYTLPPSQEEMTKNQVSLT |
| | | CLVKGFYPSDIAVEWESNGQPENNYK |
| | | TTPPVLDSDGSFFLYSRLTVDKSRWQ |
| | | EGNVFSCSVMHEALHNHYTQKSLSLS |
| | | LGK (SEQ ID NO: 12) |

Some spacer regions include all or part of an immunoglobulin (e.g., IgG1, IgG2,IgG3, IgG4) hinge region, i.e., the sequence that falls between the CH1 and CH2 domains of an immunoglobulin, e.g., an IgG4 Fc hinge or a CD8 hinge. Some spacer regions include an immunoglobulin CH3 domain or both a CH3 domain and a CH2 domain. The immunoglobulin derived sequences can include one ore more amino acid modifications, for example, 1, 2, 3, 4 or 5 substitutions, e.g., substitutions that reduce off-target binding.

An “amino acid modification” refers to an amino acid substitution, insertion, and/or deletion in a protein or peptide sequence. An “amino acid substitution” or “substitution” refers to replacement of an amino acid at a particular position in a parent peptide or protein sequence with another amino acid. A substitution can be made to change an amino acid in the resulting protein in a non-conservative manner (i.c., by changing the codon from an amino acid belonging to a grouping of amino acids having a particular size or characteristic to an amino acid belonging to another grouping) or in a conservative manner (i.c., by changing the codon from an amino acid belonging to a grouping of amino acids having a particular size or characteristic to an amino acid belonging to the same grouping). Such a conservative change generally leads to less change in the structure and function of the resulting protein. The following are examples of various groupings of amino acids: 1) Amino acids with nonpolar R groups: Alanine, Valine, Leucine, Isoleucine, Proline, Phenylalanine, Tryptophan, Methionine; 2) Amino acids with uncharged polar R groups: Glycine, Serine, Threonine, Cysteine, Tyrosine, Asparagine, Glutamine; 3) Amino acids with charged polar R groups (negatively charged at pH 6.0): Aspartic acid, Glutamic acid; 4) Basic amino acids (positively charged at pH 6.0): Lysine, Arginine, Histidine (at pH 6.0). Another grouping may be those amino acids with phenyl groups: Phenylalanine, Tryptophan, and Tyrosine.

In certain embodiments, the spacer is derived from an IgG1, IgG2, IgG3, or IgG4 that includes one or more amino acid residues substituted with an amino acid residue different from that present in an unmodified spacer. The one or more substituted amino acid residues are selected from, but not limited to one or more amino acid residues at positions 220, 226, 228, 229, 230, 233, 234, 235, 234, 237, 238, 239, 243, 247, 267, 268, 280, 290, 292, 297, 298, 299, 300, 305, 309, 218, 326, 330, 331, 332, 333, 334, 336, 339, or a combination thereof. In this numbering scheme, described in greater detail below, the first amino acid in the IgG4 (L235E,N297Q) spacer in Table 1 is 219 and the first amino acid in the IgG4 (HL-CH3) spacer in Table 1 is 219 as is the first amino acid in the IgG hinge sequence and the IgG4 hinge linker (HL) sequence in Table 1

In some embodiments, the modified spacer is derived from an IgG1, IgG2, IgG3, or IgG4 that includes, but is not limited to, one or more of the following amino acid residue substitutions: C220S, C226S, S228P, C229S, P230S, E233P, V234A, L234V, L234F, L234A, L235A, L235E, G236A, G237A, P238S, S239D, F243L, P247I, S267E, H268Q, S280H, K290S, K290E, K290N, R292P, N297A, N297Q, S298A, S298G, S298D, S298V, T299A, Y300L, V305I, V309L, E318A, K326A, K326W, K326E, L328F, A330L, A330S, A331S, P331S, I332E, E333A, E333S, E333S, K334A, A339D, A339Q, P396L, or a combination thereof.

In certain embodiments, the modified spacer is derived from IgG4 region that includes one or more amino acid residues substituted with an amino acid residue different from that present in an unmodified region. The one or more substituted amino acid residues are selected from, but not limited to, one or more amino acid residues at positions 220, 226, 228, 229, 230, 233, 234, 235, 234, 237, 238, 239, 243, 247, 267, 268, 280, 290, 292, 297, 298, 299, 300, 305, 309, 218, 326, 330, 331, 332, 333, 334, 336, 339, or a combination thereof.

In some embodiments, the modified spacer is derived from an IgG4 region that includes, but is not limited to, one or more of the following amino acid residue substitutions: 220S, 226S, 228P, 229S, 230S, 233P, 234A, 234V, 234F, 234A, 235A, 235E, 236A, 237A, 238S, 239D, 243L, 2471, 267E, 268Q, 280H, 290S, 290E, 290N, 292P, 297A, 297Q, 298A, 298G, 298D, 298V, 299A, 300L, 3051, 309L, 318A, 326A, 326W, 326E, 328F, 330L, 330S, 331S, 331S, 332E, 333A, 333S, 333S, 334A, 339D, 339Q, 396L, or a combination thereof, wherein the amino acid in the unmodified spacer is substituted with the above identified amino acids at the indicated position.

For amino acid positions in immunoglobulin discussed herein, numbering is according to the EU index or EU numbering scheme (Kabat et al. 1991 Sequences of Proteins of Immunological Interest, 5th Ed., United States Public Health Service, National Institutes of Health, Bethesda, hereby entirely incorporated by reference). The EU index or EU index as in Kabat or EU numbering scheme refers to the numbering of the EU antibody (Edelman et al. 1969 Proc Natl Acad Sci USA 63:78-85).

A variety of transmembrane domains can be used in the CAR. Table 2 includes examples of suitable transmembrane domains. Where a spacer domain is present, the transmembrane domain is located carboxy terminal to the spacer domain.

TABLE 2
Examples of Transmembrane Domains

| Name | Accession | Length | Sequence |
|---|---|---|---|
| CD3z | J04132.1 | 21 aa | LCYLLDGILFIYGVILTALFL |
| | | | (SEQ ID NO: 13) |
| CD28 | NM_006139 | 27 aa | FWVLVVVGGVLACYSLLVTVA |
| | | | FIIFWV(SEQ ID NO: 14) |
| CD28(M) | NM_006139 | 28 aa | MFWVLVVVGGVLACYSLLVTVA |
| | | | FIIFWV(SEQ ID NO: 15) |
| CD4 | M35160 | 22 aa | MALIVLGGVAGLLLFIGLGIFF |
| | | | (SEQ ID NO: 16) |
| CD8tm | NM_001768 | 21 aa | IYIWAPLAGTCGVLLLSLVIT |
| | | | (SEQ ID NO: 17) |
| CD8tm2 | NM_001768 | 23 aa | IYIWAPLAGTCGVLLLSLVI |
| | | | TLY(SEQ ID NO: 18) |
| CD8tm3 | NM_001768 | 24 aa | IYIWAPLAGTCGVLLLSLVI |
| | | | TLYC(SEQ ID NO: 19) |
| 41BB | NM_001561 | 27 aa | IISFFLALTSTALLFLLFF |
| | | | LTLRFSVV(SEQ ID NO: 20) |

Many of the CAR described herein include one or more (e.g., two) costimulatory domains. The costimulatory domain(s) are located between the transmembrane domain and the CD3ζ signaling domain. Table 3 includes examples of suitable costimulatory domains together with the sequence of the CD3ζ signaling domain.

TABLE 3
CD3ζ Domain and Examples of Costimulatory Domains

| Name | Accession | Length | Sequence |
|---|---|---|---|
| CD3ζ | J04132.1 | 113 aa | RVKFSRSADAPAYQQGQNQLYNELNL |
| | | | GRREEYDVLDKRRGRDPEMGGKPRRK |
| | | | NPQEGLYNELQKDKMAEAYSEIGMKG |
| | | | ERRRGKGHDGLYQGLSTATKDTYDAL |
| | | | HMQALPPR(SEQ ID NO: 21) |
| CD28 | NM_006139 | 42 aa | RSKRSRLLHSDYMNMTPRRPGPTRKH |
| | | | YQPYAPPRDFAAYRS |
| | | | (SEQ ID NO: 22) |
| CD28gg* | NM_006139 | 42 aa | RSKRSRGGHSDYMNMTPRRPGPTRKH |
| | | | YQPYAPPRDFAAYRS |
| | | | (SEQ ID NO: 23) |
| 41BB | NM_001561 | 42 aa | KRGRKKLLYIFKQPFMRPVQTTQEED |
| | | | GCSCRFPEEEEGGCEL |
| | | | (SEQ ID NO: 24) |
| OX40 | | 42 aa | ALYLLRRDQRLPPDAHKPPGGGSFRT |
| | | | PIQEEQADAHSTLAKI |
| | | | (SEQ ID NO:25) |

1. A method for producing a T cell population expressing a recombinant T cell receptor, comprising providing a population of T cells harboring a vector encoding a recombinant T cell receptor, culturing the population of T cells in growth media under conditions and for a time to expand the population of T cells wherein the growth media comprises an inhibitor of Akt activity.

2. The method of claim 1 wherein the Akt inhibitor is added to the growth media during the culturing step.

3. The method of claim 1 wherein the Akt inhibitor is sufficient to reduce the Akt 1 or Akt 2 activity or both by at least 25%.

4. The method of claim 1 wherein the Akt inhibitor inhibits Akt1 and Akt2 with an IC₅₀ less than 1000 nM.

5. The method of claim 1 wherein the Akt inhibitor is selected from the group consisting of: Akt Inhibitor VIII (1,3-dihydro-1-[1-[[4-(6-phenyl-1H-imidazo[4,5-g]quinoxalin-7-yl)phenyl]methyl]-4-piperidinyl]-2H- benzimidazol-2-one), Akt Inhibitor X (2-chloro-N,N-diethyl-10H-phenoxazine-10-butanamine, monohydrochloride), MK-2206 (8-(4-(1-aminocyclobutyl)phenyl)-9-phenyl-[1,2,4]triazolo[3,4-f][1,6]naphthyridin-3(2H)-one), uprosertib (N-((S)-1-amino-3-(3,4-difluorophenyl)propan-2-yl)-5-chloro-4-(4-chloro-1-methyl-1H-pyrazol-5-yl)furan-2-carboxamide), ipatasertib ((S)-2-(4-chlorophenyl)-1-(4-((5R,7R)-7-hydroxy-5-methyl-6,7-dihydro-5H-cyclopenta[d]pyrimidin-4-yl)piperazin-1-yl)-3- (isopropylamino)propan-1-one), AZD 5363 (4-Piperidinecarboxamide, 4-amino-N-[(1S)-1-(4-chlorophenyl)-3-hydroxypropyl]-1-(7H-pyrrolo[2,3-d]pyrimidin-4-yl)), perifosine, GSK690693, GDC-0068, tricirbine, CCT128930, A-674563, PF-04691502, AT7867, miltefosine, PHT-427, honokiol, triciribine phosphate, KP372-1A (10H-indeno[2,1-e]tetrazolo[1,5-b][1,2,4]triazin-10-one) H-8, H-89, NL-71-101, 7-azaindole, 3-aminopyrrolidine, ipatasertib, A-443654, AT13148, afuresertib (GSK2110183), DC120, edelfosine (1-O-octadecyl-2-O-methyl-rac-glycero-3-phosphocholine, ET-18-OCH₃), ilmofosine (BM 41.440), erucylphosphocholine (ErPC), erufosine (ErPC3, erucylphosphohomocholine), indole-3-carbinol, 3-chloroacetylindole, diindolylmethane, SR13668 (diethyl 6-methoxy-5,7-dihydroindolo [2,3-b]carbazole-2,10-dicarboxylate), OSU-A9, PH-316, PIT-1, PIT-2, DM-PIT-1, N-[(1-methyl-1H-pyrazol-4-yl)carbonyl]-N′-(3-bromophenyl)-thiourea), TCN-P, API-1, ARQ 092, BAY 1125976, 3-methyl-xanthine, quinoline-4-carboxamide, 2-[4-(cyclohexa-1,3-dien-1-yl)-1H-pyrazol-3-yl]phenol, 3-oxo-tirucallic acid, acetoxy-tirucallic acid; lactoquinomycin, frenolicin B, kalafungin, medermycin, Boc-Phe-vinyl ketone, and 4-hydroxynonenal (4-HNE).

6. The method of claim 1 wherein the growth media comprises IL-2.

7. The method of claim 1 wherein recombinant T cell receptor is an engineered TCR or a chimeric antigen receptor (CAR).

8. The method of claim 1 wherein the step of providing a population of T expressing a recombinant T cell receptor comprises:
obtaining T cells from the patient or obtaining T cells allogenic to the patient, treating the obtained T cells to isolate a population of cells enriched for central memory T cells, and
transducing at least a portion of the isolated population of cells with a viral vector comprising an expression cassette encoding a chimeric antigen receptor.

9. The method of claim 1 wherein the step of providing a population of T cells expressing a recombinant T cell receptor comprises:
obtaining T cells from the patient or obtaining T cells allogenic to the patient,
treating the obtained T cells to isolate a population of cells enriched for CD8+ T cells, and
transducing at least a portion of the isolated population of cells with a viral vector comprising an expression cassette encoding a chimeric antigen receptor.

10. The method of claim 1 wherein the recombinant T cell receptor is a chimeric antigen receptor (CAR) comprises:
a target binding domain;
a transmembrane domain selected from: a CD4 transmembrane domain or variant thereof having 1-10 amino acid modifications, a CD8 transmembrane domain or variant thereof having 1-10 amino acid modifications, a CD28 transmembrane domain or a variant thereof having 1-10 amino acid modifications, and a CD3ζ transmembrane domain or a variant thereof having 1-10 amino acid modifications;
a costimulatory domain; and
a CD3ζ signaling domain or a variant thereof having 1-10 amino acid modifications.

11. The method of claim 10 wherein the costimulatory domain is selected from the group consisting of: a CD28 costimulatory domain or a variant thereof having 1-10 amino acid modifications, a 4IBB costimulatory domain or a variant thereof having 1-10 amino acid modifications and an OX40 costimulatory domain or a variant thereof having 1-10 amino acid modifications.

12. The method of claim 11 wherein the chimeric antigen receptor comprises two different costimulatory domains selected from the group consisting of: a CD28 costimulatory domain or a variant thereof having 1-10 amino acid modifications, a 4IBB costimulatory domain or a variant thereof having 1-10 amino acid modifications and an OX40 costimulatory domain or a variant thereof having 1-10 amino acid modifications.

13. The method of claim 11 wherein the chimeric antigen receptor comprises two different costimulatory domains selected from the group consisting of: a CD28 costimulatory domain or a variant thereof having 1-2 amino acid modifications, a 4IBB costimulatory domain or a variant thereof having 1-2 amino acid modifications and an OX40 costimulatory domain or a variant thereof having 1-2 amino acid modifications.

14. The method of claim 13 wherein the chimeric antigen receptor comprises: a transmembrane domain selected from: a CD4 transmembrane domain or variant thereof having 1-2 amino acid modifications, a CD8 transmembrane domain or variant thereof having 1-2 amino acid modifications, a CD28 transmembrane domain or a variant thereof having 1-2 amino acid modifications, and a CD3ζ transmembrane domain or a variant thereof having 1-2 amino acid modifications; a costimulatory domain; and CD3ζ signaling domain of a variant thereof having 1-2 amino acid modifications.

15. The method of claim 10 wherein the chimeric antigen receptor comprises a spacer region located between the target binding domain and the transmembrane domain.

16. The method of claim 10 wherein the target binding domain is a scFV.

17. The method of claim 16 wherein the scFv binds a tumor cell antigen.

18. The method of claim 1 wherein the step of providing a population of T cells harboring a vector encoding a recombinant T cell receptor comprising activating a population of T cells and transducing the activated T cells with a vector encoding a recombinant T cell receptor, wherein the activation step and the transduction step occur in the presence of an Akt inhibitor.

19. The method of claim 1 wherein the T cells comprise: αβ T cells, γδ T cells, NK T cells or a combination thereof.

20. (canceled)

21. (canceled)


SEQUENCE LISTING (25 sequences)

SEQ ID NO: 1 - skipped

SEQ ID NO: 2 - protein, 10 aa, synthetic construct
GGGSSGGGSG

SEQ ID NO: 3 - protein, 12 aa, synthetic construct
ESKYGPPCPPCP

SEQ ID NO: 4 - protein, 12 aa, Homo sapiens
ESKYGPPCPSCP

SEQ ID NO: 5 - protein, 22 aa, synthetic construct
ESKYGPPCPPCPGGGSSGGGSG

SEQ ID NO: 6 - protein, 39 aa, Homo sapiens
IEVMYPPPYLDNEKSNGTIIHVKGKHLCPSPLFPGPSKP

SEQ ID NO: 7 - protein, 48 aa, Homo sapiens
AKPTTTPAPRPPTPAPTIASQPLSLRPEACRPAAGGAVHTRGLDFACD

SEQ ID NO: 8 - protein, 45 aa, Homo sapiens
TTTPAPRPPTPAPTIASQPLSLRPEACRPAAGGAVHTRGLDFACD

SEQ ID NO: 9 - protein, 129 aa, synthetic construct
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 229 aa, synthetic construct
ESKYGPPCPSCPAPEFEGGPSVFLFPPKPKDTLMISRTPEVTCVVVDVSQEDPEVQFNWYVDGVEVHQAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 229 aa, synthetic construct
ESKYGPPCPPCPAPEFEGGPSVFLFPPKPKDTLMISRTPEVTCVVVDVSQEDPEVQFNWYVDGVEVHQAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 21 aa, Homo sapiens
LCYLLDGILFIYGVILTALFL

SEQ ID NO: 14 - protein, 27 aa, Homo sapiens
FWVLVVVGGVLACYSLLVTVAFIIFWV

SEQ ID NO: 15 - protein, 28 aa, synthetic construct
MFWVLVVVGGVLACYSLLVTVAFIIFWV

SEQ ID NO: 16 - protein, 22 aa, Homo sapiens
MALIVLGGVAGLLLFIGLGIFF

SEQ ID NO: 17 - protein, 21 aa, Homo sapiens
IYIWAPLAGTCGVLLLSLVIT

SEQ ID NO: 18 - protein, 23 aa, Homo sapiens
IYIWAPLAGTCGVLLLSLVITLY

SEQ ID NO: 19 - protein, 24 aa, Homo sapiens
IYIWAPLAGTCGVLLLSLVITLYC

SEQ ID NO: 20 - protein, 27 aa, Homo sapiens
IISFFLALTSTALLFLLFFLTLRFSVV

SEQ ID NO: 21 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 41 aa, Homo sapiens
RSKRSRLLHSDYMNMTPRRPGPTRKHYQPYAPPRDFAAYRS

SEQ ID NO: 23 - protein, 41 aa, synthetic construct
RSKRSRGGHSDYMNMTPRRPGPTRKHYQPYAPPRDFAAYRS

SEQ ID NO: 24 - protein, 42 aa, Homo sapiens
KRGRKKLLYIFKQPFMRPVQTTQEEDGCSCRFPEEEEGGCEL

SEQ ID NO: 25 - protein, 42 aa, Homo sapiens
ALYLLRRDQRLPPDAHKPPGGGSFRTPIQEEQADAHSTLAKI